(19) United States
(12) Patent Application Publication
(10) Pub. No.: US 2011/0275153 A1
(43) Pub. Date: Nov. 10, 2011

(54) CRYOGENIC STORAGE DEVICE

(75) Inventors: Bradley K. Butler, Spencer, IN (US); Troy W. Wingler, Martinsville, IN (US); James M. Carlson, Bloomington, IN (US); Scott K. Philhower, Bloomington, IN (US)

(73) Assignee: Vance Products Inc., d/b/a Cook Urological Inc., Spencer, IL (US)

(21) Appl. No.: 13/101,586

(22) Filed: May 5, 2011

Related U.S. Application Data
(60) Provisional application No. 61/332,005, filed on May 6, 2010.

(51) Int. Cl. C12N 5/071; C12M 3/00

(52) U.S. Cl. 435/374; 435/307.1

(57) ABSTRACT

A cryopreservation device for storing reproductive biological material is provided. The device comprises an elongate first member includes an elongate first member extending between a distal end and a proximal end, a first bulge portion disposed around a circumference of the first member, and a trough defined within the first member, the trough being configured to receive a reproductive biological sample thereon. A second member includes a second member with a lumen defined therethrough. The second member is configured to slide over the first member and the inner diameter of the second member is similar to an outer diameter of the bulge portion to form a seal between the first and second members.

### CROSS-REFERENCE TO RELATED APPLICATIONS

This application claims priority from U.S. Provisional Application No. 61/332,005, filed on May 6, 2010, the entirety thereof is hereby full incorporated by reference herein.

### TECHNICAL FIELD

This disclosure relates to devices suitable to receive and enclose a biological sample for long term cryogenic storage. Cryogenic storage is often used for the purpose of halting biological activity in cells such that a biological sample can be stored in situations where it is not possible or convenient to manipulate the sample at the present time. For example, in situations where patients are undergoing in vitro fertilization procedures, oocytes are often harvested from a patient who has undergone hormone treatment to cause their ovaries to produce a larger number of follicles and oocytes that normally produced during a typical cycle. While the hormone therapy is needed to stimulate the production of multiple oocytes, the same hormone therapy often causes side affects to the patient's uterine tissue or other portions of their reproductive tissue that minimizes the chances of successful embryo implantation into the patient during the same cycle.

Accordingly, it is often desired to fertilize oocytes to create embryos at the time they are harvested and cryogenically store them for implantation during a future cycle. In other situations it is desirable for female patients to cryogenically store harvested and unfertilized oocytes for potential future use. Other types of biological tissue is often desired to be stored on a long term basis for future research or therapeutic purposes, such as muscle tissue. Biological samples to be cryogenically stored are stored with vitrification media that is used to prepare the sample for long term storage, which may include removing water from the sample. It is known that the vitrification media is harmful to the biological sample at room temperature so it is desired to minimize the time that the biological sample is at room temperature in the presence of vitrification media.

### BRIEF SUMMARY

A first representative embodiment of the disclosure provides a method of cryogenically preserving biological material. The method includes the steps of providing a first elongate member with a trough disposed upon an outer surface of the first elongate member, and a first bulge coaxially defined upon the outer surface with an outer diameter of the first bulge larger than an outer diameter of the first member. A biological material is deposited upon the trough. The method further includes the step of sliding an elongate second member over the first member to form a preservation assembly. The second member has an inner diameter substantially the same as the outer diameter of the first bulge such that an inner surface of the second member makes substantially continuous contact around the circumference of the bulge portion. The method further includes the step of depositing the preservation assembly within a cryogenic medium.

A second representative embodiment of the disclosure provides a cryopreservation device for storing reproductive biological material configured to receive a biological sample for long term cryogenic storage. The device includes an elongate first member extending between a distal end and a proximal end and a first bulge portion disposed around a circumference of the first member, and a trough defined within the first member. A second member with a lumen is defined therethrough, the second member is configured to slide over the first member, the inner diameter of the second member being substantially the same as an outer diameter of the bulge portion to form a seal between the first and second members.

A third representative embodiment of the disclosure provides a cryopreservation device for storing reproductive biological material configured to receive a biological sample for long term storage. The device includes an elongate first member extending between a distal end and a proximal end and a first bulge portion disposed around a circumference of the first member upon the distal end, and a trough defined within the first member. The proximal end of the first member comprises a flared portion that includes an increasing outer diameter along the length thereof in a direction extending away from the trough. A second member with a lumen is defined therethrough, the second member is configured to slide over the first member, the inner diameter of the second member being substantially the same as an outer diameter of the bulge portion to form a seal between the first and second members.

Advantages of the present disclosure will become more apparent to those skilled in the art from the following description of the preferred embodiments of the disclosure that have been shown and described by way of illustration. As will be realized, the disclosed subject matter is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a perspective view of a device for cryogenic storage of a biological sample with the sheath enclosing the trough.

FIG. 1A is the perspective view of FIG. 1 showing the sheath disposed over the proximal end of the shuttle with the trough exposed.

FIG. 2 is an exploded view of the device of FIG. 1.

FIG. 3 is a side view of the shuttle of FIG. 1.

FIG. 3A is a detail view of detail A of FIG. 3.

FIG. 4 is a cross-sectional view depicting engagement between the sheath and the bulge portions upon the shuttle and registration between the central portion of the sheath and the trough.

FIG. 5 is the view of FIG. 4 depicting the central portion crimped for contact with a biological sample disposed upon the trough of the shuttle.

FIG. 6 is a side view of an alternate shuttle usable with the device for cryogenic storage of a biological sample.

FIG. 7 is a cross-sectional view of the shuttle of FIG. 6 surrounded by a sheath.

FIG. 8 is a side view of another alternate shuttle usable with the device for cryogenic storage of a biological sample.

FIG. 9 is a cross-sectional view of an alternate sheath usable with the shuttle of FIG. 8.

### DETAILED DESCRIPTION OF THE DRAWINGS AND THE PRESENTLY PREFERRED EMBODIMENTS

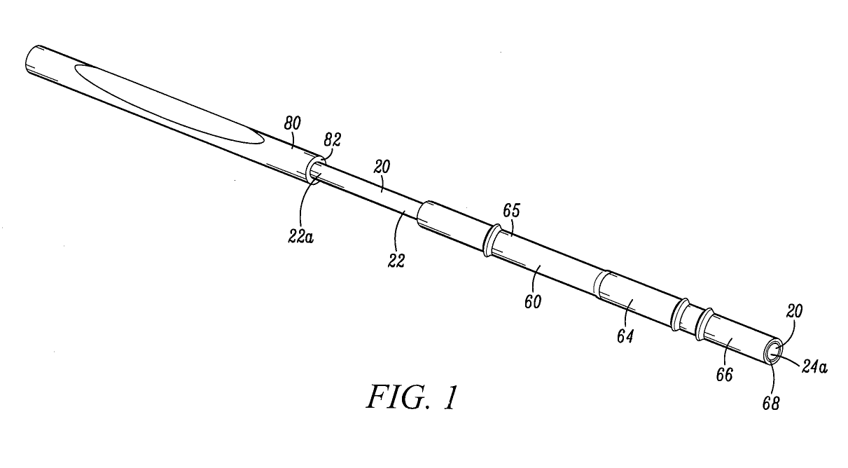

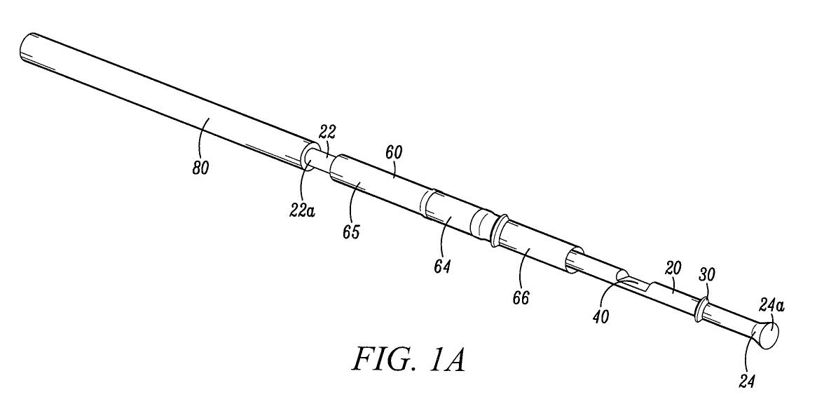

Turning now to FIGS. 1-5, a device 10 for cryogenically preserving biological matter is provided. The device 10 is configured to receive and enclose a volume of biological material within the device such that substantially no leakage occurs (either biological material leaking from within the device 10, or foreign liquids or gasses leaking into the internal portion of the device 10). The device 10 is also configured to allow the user to establish the substantially leak proof configuration without any external tools, or an external heat source, and further disestablish the leak proof configuration to allow for removal of the biological matter without any tools. The device 10 further allows for receipt of a biological sample, assembly of the device 10 and placement of the device within a cryogenic bath in a relatively short time period.

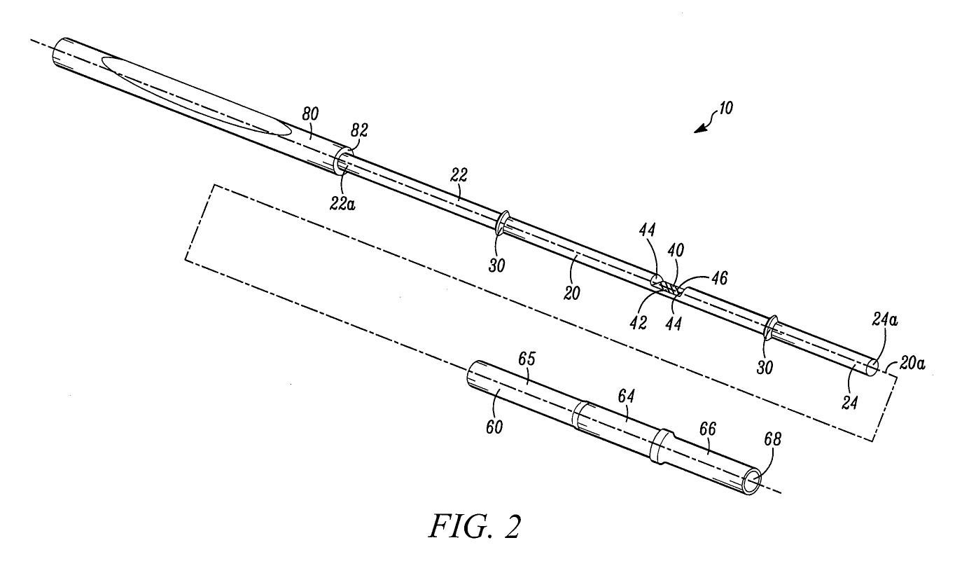

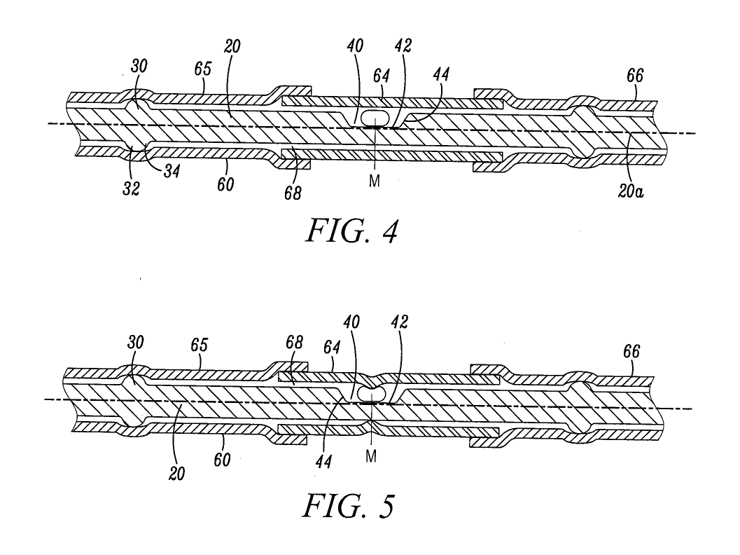

The device 10 includes an elongate shuttle 20 and an elongate sheath 60 that may be slidably disposed over the outer surface of the shuttle 20. The sheath 20 may be an elongate rod that extends between a distal end portion 24 and a proximal end portion 22 with a longitudinal axis 20a disposed therethrough. The shuttle 20 further includes a trough 40 that provides a portion of the outer surface of the shuttle 20 that extends within the cylindrical surface that forms the majority of the outer surface of the shuttle 20. The trough 40 may include a bottom surface 42 that is configured to receive a biological sample M (such as a reproductive biological sample, e.g. embryo or oocyte in a drop with vitrification media, or such as other types of cellular material suitable for long term storage, such as muscle tissue and the like) (FIG. 4) thereon, and two end surfaces 44 that provide the transition between the cylindrical remainder of the shuttle 20 and the bottom surface 42 of the trough 40. In some embodiments, the bottom surface 42 may include a plurality of surface features 46 (FIG. 2), such as grooves, indentations, blind holes, or the like. The surface features 46 are configured to provide a relatively strong bond, connection, or a relatively high frictional surface such that the biological sample M placed upon the bottom surface 42 of the trough 40 is retained thereon regardless of the orientation of the device 10, or upon the receipt of vibrational or impact forces upon the device 10.

In some embodiments, the two end surfaces 44 of the trough 40 may be disposed at an acute angle, such as about 45 degrees, with respect to the longitudinal axis 20a of the shuttle 20. The formation of acute end surfaces 44 allows for ease of manipulating a biological sample M positioned proximate one end of the bottom surface 42 because it allows an oblique angle of attack to position the biological sample M with tweezers, a needle, pipet, or other suitable instrument appropriate for manipulating the biological sample M.

The trough 40 may be configured to receive a reproductive biological sample M thereon, such as a fertilized embryo that is embedded in a drop of vitrification media, or an oocyte that may be embedded in suitable vitrification media. A typical reproductive biological sample M may have a volume of 0.5 micro liters, which has a nominal diameter of about 0.5 mm (0.02 inches). to about 1 mm (0.04 inches). In some embodiments, the trough 40 is configured such that a nominally sized biological sample M may be disposed upon the trough 40 with the upper surface of the biological sample M (i.e. the point(s) upon the biological sample M that is furthest away from the bottom surface 42 of the trough 40) below the outer surface of the shuttle 20. In other words, the trough 40 is configured such that a nominal sample M completely fits within the void created within the trough 40. In other embodiments, the trough 40 may be oriented such that the outer surface (as defined above) of the biological sample M is disposed below the central portion 32 of the bulge portion 30 (discussed in detail below). In still other embodiments, the trough 40 is configured such that an upper surface of a nominal biological sample M is disposed below the inner diameter of the end portions 65, 66 of the sheath 60. In the these embodiments, the trough 40 is configured such that a nominal biological sample M placed thereon is not disturbed when the sheath 60 is initially slid over and removed from the shuttle 20.

The shuttle 20 additionally includes two or more bulge portions 30 that are disposed upon the outer circumferential surface of the shuttle 20. The bulge portions 30 each include an outer diameter that is slightly larger than the outer diameter of neighboring portion of the shuttle 20, and in some embodiments, the outer diameter of the remaining portions of the shuttle 20. In some embodiments, the bulge portion 30 may include an arcuate outer profile from one side of the bulge to the other, such that a central portion 32 of the bulge portion 30 has a larger outer diameter than side portions 34 on opposite sides of the central portion 32. In some embodiments, the outer profile of the bulge portion 30 is the same around the entire circumferential surface of the shuttle 20. In other embodiments, the outer profile of the bulge portion 30 varies around the circumference of the shuttle 20.

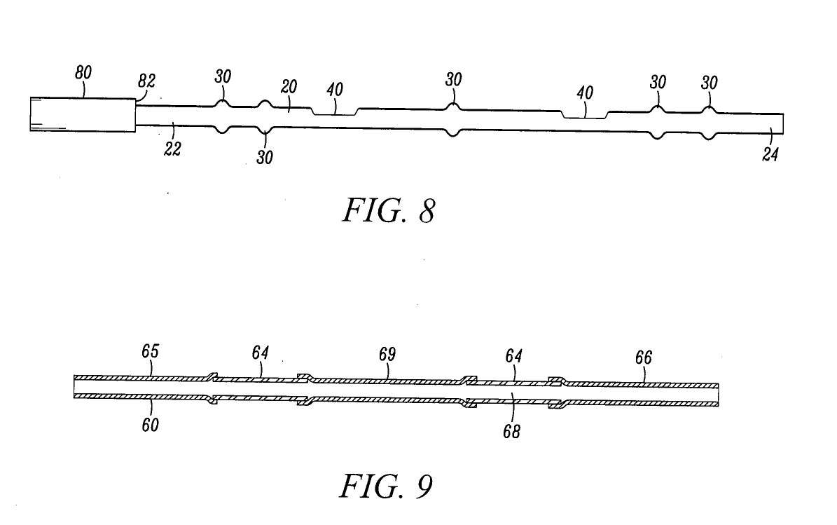

In some embodiments the two bulge portions 30 are disposed with the same size and shape while in other embodiments the opposite bulge portions 30 may have differing sizes and/or shapes. In some embodiments, a single bulge portion 30 may be disposed upon the shuttle on each side of the trough 40, while in other embodiments shown in FIG. 8, two or more bulge portions 30 may be disposed in series on each opposite side of the trough 40 in order to provide additional points of contact between the bulge portion 30 and the inner surface of the sheath 60, as discussed below, for redundancy purposes. In some embodiments shown in FIG. 8, two or more troughs 40 may be disposed upon a single shuttle 20. In these embodiments, bulge portions 30 may be provided on upon the shuttle 20 upon the outer edges of the series of troughs 40 (with FIG. 8 depicting two bulge portions 30, while a single bulge portion 30 is also contemplated), while in some embodiments, bulge portions 30 may additionally be provided between the neighboring troughs 40 to minimize or eliminate fluid communication between neighboring troughs 40 when the device 10 is assembled, as discussed herein.

The bulge portions 30 may be formed monolithically with the remainder of the shuttle 20, while in other embodiments, the bulge portion 30 may be a separate component from the shuttle 20 that is fixed thereto, either through friction or snap fit, adhesive, or by otherwise mechanically fixing thereto. In embodiments where the bulge 30 is a separate component from the shuttle 20, the bulge 30 may be one or more o-rings that are received upon the outer surface of the shuttle 20, or within an arcuate slot defined upon the surface of the shuttle 20. In some embodiments, the shuttle 20 (with or without the handle 80) including the trough 40 and bulge portions 30 is a single molded piece, or may be machined, or otherwise formed from a single stock of material.

In some embodiments, the shuttle 20 may be about 5 inches long, or within a range (and inclusive of the lengths within the range) of about 3 to about 7 inches, or other appropriate lengths (either inclusive of a handle 80 fixed to the proximal tip 22a of the shuttle 20, or exclusive of the length of the handle 80). The length of the shuttle 20 may be configured to be long enough to be easily manipulated by the user, easy to identify and manipulate when placed within a liquid nitrogen bath (or other cryogenic liquid or cryogenic container), and provide a suitably sized trough 40 for receipt of a biological sample M thereon.

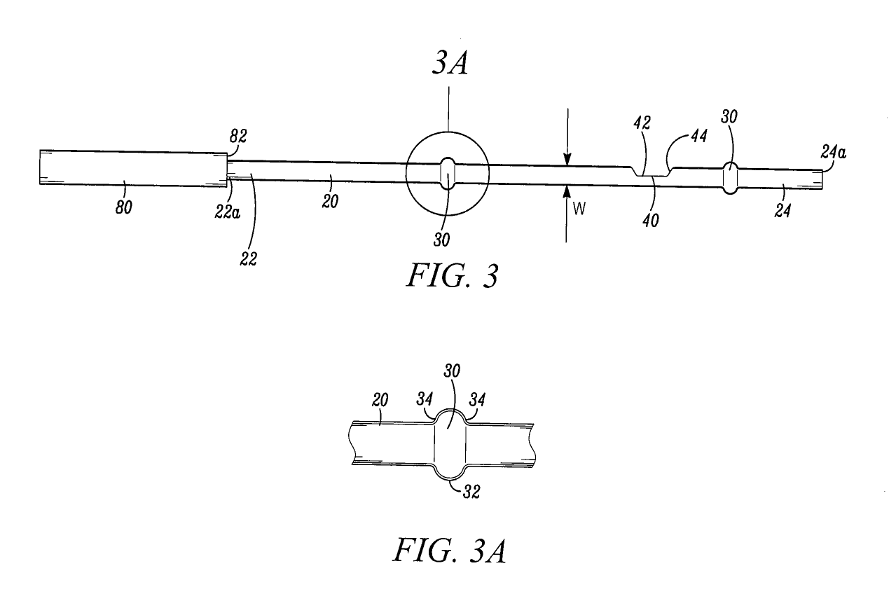

The shuttle 20 may have an outer diameter W (FIG. 3) between about 0.05 and 0.1 inches, or in some embodiments between 0.06 and 0.08 inches. In some exemplary embodiments, the outer diameter W may be 0.06, 0.065, 0.068, 0.07, 0.075, and 0.08 inches. The outer diameter W is sized to provide sufficient strength and rigidity to the shuttle 20, and to provide an adequately sized trough 40 while maintaining sufficient strength and rigidity of the entire shuttle 20. In some embodiments, the trough 40 may be disposed such that the bottom surface 42 of the trough extends through, or is proximate to the longitudinal axis 20a of the shuttle 20, i.e. that the trough 40 extends through about half of the thickness of the shuttle 20. In a representative embodiment where the outer diameter for the shuttle 20 is 0.068 inches, the bottom surface 42 of the trough 40 is 0.034 inches from the outer surface of the shuttle 20. The trough 40 is sized to provide sufficient space for various types of biological samples M to be disposed thereon, while minimizing the size of the trough 40 to maintain adequate stiffness and column strength of the shuttle 20. In some embodiments, the trough may be about 0.25 inches wide, about 0.3 inches wide, or other similar suitable widths.

The bulge portions 30 are configured with an outer diameter, or at least a portion with an outer diameter at least slightly larger than the outer diameter than the remainder of the shuttle 20. Similarly, in some embodiments, the outer diameter (or at least the largest outer diameter) of the bulge portion 30 may be substantially the same as the inner diameter of the sheath 60. As discussed below, in embodiments where the sheath 60 is formed from both a central shape memory portion 64 and first and second substantially flexible end portions 65, 66 on opposite sides of the central portion 64, the inner diameter of the central portion 64 may be slightly larger than the largest outer diameter of the bulge portion 30, while the inner diameter of the end portions 65, 66 may be substantially the same as, or in other embodiments slightly smaller than, the largest outer diameter of the bulge portion 30. In a first exemplary embodiment where the diameter of the shuttle 20 is 0.068 inches, the outer diameter of the bulge portions 30 (or the largest outer diameter of the bulge portions 30) is 0.088 inches, and in another embodiment the largest outer diameter of the bulge portion is 0.079 inches. In some embodiments, the shuttle 20 may be made from PEEK (polyether ether ketone) or other plastic or thermoplastic.

The sheath 60 is an elongate member that extends from a distal end 61 to a proximal end 62 with a lumen 68 disposed therethrough. The sheath 60 is configured to slide over the shuttle 20 coaxially. As mentioned above, the inner diameter of the lumen 68, or at least a portion of the lumen 68 is substantially the same, or in some embodiments slightly smaller than the outer diameter of the bulge portion 30. Specifically, in some embodiments, the inner diameter of each of the first and second ends 65, 66 may be less than the inner diameter of the central portion 64, and the inner diameter of each or one of the first and second ends 65, 66 may be less than at least the largest outer diameter of the bulge portion 30.

In some embodiments, the sheath 60 may be formed with a central portion 64 and opposite end portions 65, 66. The central portion 64 may be made from a shape memory material, such as Nitinol. The central portion 64 may be substantially tubular in its memorized austenite orientation and configured with an inner diameter just larger than the largest outer diameter of the bulge portion 30 such that the central portion 64 of the sheath 60 can easily slide over the shuttle 20. The shape memory material or alloy selected may be one with a martensite to austenite transition temperature located at about room temperature (e.g. 70-75 degrees). In other embodiments, the alloy may be selected to have a transition temperature at a temperature above normal room temperature but below normal body temperature. Pending U.S. Published Application Number 2009/0123992 titled “Shape Shifting Vitrification Device” issued as U.S. patent Ser. No. ______ includes a description of the operation of shape memory materials, such as Nitinol, and the application is hereby fully incorporated by reference herein.

The sheath 60 may be configured such that the central portion 64 is disposed in registry with the trough 40 of the shuttle 20 when the sheath 60 is slid over and aligned with the shuttle 20. In embodiments shown in FIG. 8 where two or more troughs 40 are disposed upon a single shuttle 20, the sheath 60 may include a similar number of central portions 64 (which may be separated by intermediate flexible portions 69 similar to end portions 65, 66) that are aligned in registry with the multiple troughs 40 when the device 10 is assembled, as shown in FIG. 9. In other embodiments, the sheath 60 may include a single central portion 64 that is of sufficient length to be placed in registry with each of the two or more troughs 40 disposed upon the shuttle 20, and the central portion 64 is configured to be simultaneously crimped in multiple locations, as discussed below. In some embodiments, the shuttle 20 may be provided with markings that allow for precise positioning of the sheath 60 over the shuttle 20 for registration of the central portion 64 and the trough 40 (or central portions 64 and troughs 40).

As discussed below, the registration between the central portion 64 and the trough 40, along with markings that may be provided upon the outer surface of the central portion 64, allows precise crimping or otherwise inward deformation such that the inner surface of the central portion 64 contacts the biological sample M disposed upon the bottom surface 42 of the trough 40. The deformation of the central portion 64 and contact with the biological sample M allows direct conduction heat transfer between the biological sample M and the central portion 64, which provides for extremely rapid cool down of the biological sample M and device 10 when the device 10 is cooled in a cryogenic bath, and extremely rapid heat up of the sample M and the device 10 when the device 10 is placed in a warming bath. A detailed description of the process of crimping a portion similar to the central portion 64 is found in the U.S. 2009/0123992 published application. The placement of the sheath 60 over and in registration with the shuttle 20 establishes the preservation assembly.

In some embodiments, tweezers may be used to crimp or deform the central portion inwardly, and in some embodiments, metered tweezers or pliers, i.e. devices with two opposed jaws that are configured to be moved to a position with the tips of the jaws a specific distance apart but not touch, may be used to crimp the central portion 64 to a specific geometry that allows contact with a range of potential sizes of biological samples but prevents contact between the central portion 64 and the shuttle 20 (which could damage the shuttle 20) may be used.

The inner surface of the central portion 64 (and potentially the inner surface of the end portions 65, 66) may be hydrophobic (either intrinsically hydrophobic or coated with a hydrophobic material) to prevent the biological sample M from sticking to the inner surface of the sheath 60. Hydrophobic coatings such as polytetrafluoroethylene or polyxylene polymer may be applied to the inner surface of the central portion 64. FEP, which may be used to make the first and second end portions 65, 66 is hydrophobic. In embodiments where the first and second end portions 65, 66 are made from other plastics or other flexible materials, the inner surface thereof may have a hydrophobic coating.

The first and second end portions 65, 66 of the sheath 60 are each fixed to opposite ends of the central member 64. The first and second ends 65, 66 may be made from a relatively flexible material, such as FEP (Fluorinated ethylene propylene) or other flexible materials that are configured to withstand the very low cryogenic temperatures as well as tolerate the rapid cool down and heat up rates as the device 10 is placed in a cryogenic fluid as well as a warming bath. The materials are also configured to expand and contract about the same amount as the material chosen for the shuttle 20 during the large thermal changes associated with vitrification and subsequent return to the warmed temperature. The material chosen for the end portions 65, 66 of the sheath 60 preferably has a lower durometer than the material chosen for the shuttle 20, such that the end portions 65, 66 of the sheath 60 deform when the sheath 60 engages the shuttle 20.

The first and second ends 65, 66 of the sheath 60 may be fixed to opposite ends of the central portion 64 with adhesive, with a crimped connection, or with other connection methods known in the art. In some embodiments best shown in FIGS. 2, 4, and 5, the first and second ends 65, 66 may be friction fit to the central portion 64 with the inner surface of the ends of the first and second ends 65, 66 resting upon the outer surface of the central portion 64. The first and second ends 65, 66 may be fixed to the central portion 64 by heating the first and second ends 65, 66 to a temperature where the material defining the first and second ends 65, 66 becomes increasingly flexible and manually flaring the end portions of the first and second ends to an inner diameter larger than the outer diameter of the central portion 64, such that the first and second ends 65, 66 may be positioned coaxially around the end portions of the central portion 64. As the material of the first and second ends 65, 66 cools, the material contracts toward its original dimensions, which establishes a tight bond between the central portion 64 and the respective first and second end portion 65, 66.

The first and second ends 65, 66 are configured to be sufficiently flexible around room temperature to allow sheath 60 to slide over the shuttle 20, even in embodiments wherein the inner diameter of the first and second ends 65, 66 is slightly smaller than at least the largest outer diameter of the bulge portion 30. The radial expansion of the sheath 60 over the bulge portion 30 causes a tight seal between the respective end portion 65, 66 and the respective bulge 30 around the circumference of the shuttle 20, which prevents passage of fluid therebetween. The end portions 65, 66 of the sheath 60 are configured to be sufficiently flexible to allow the user to slide the sheath 60 over the shuttle 20 with one of their hands, while the user holds the shuttle 20 steady in their other hand.

In some embodiments best shown in FIGS. 1 and 1A, the shuttle 20 is significantly longer than the sheath 60 and the proximal end portion 22 of the shuttle 20, and specifically the distance between the proximal end or step 82 and the trough 40 is longer than the length of the sheath 60. This length relationship as depicted in FIG. 1A, allows the sheath 60 to be positioned upon the proximal end portion 22 of the shuttle 20 with the trough 40 exposed for convenient placement of a biological sample thereon, with the shuttle being easily slid along the shuttle 20 until the central portion 64 of the sheath 60 is in registry with the trough 40. In some embodiments, the device 10 may be packaged and shipped to the user in the configuration shown in FIG. 1A, such that the user can place a biological sample M upon the trough 40 and properly align the sheath 60 with respect to the shuttle 20 in an easy and rapid process.

In some embodiments specifically shown in FIG. 1A, the distal tip 24 of the distal end portion 24 of the shuttle 20 may be flared outward. The flared distal tip 24 is configured to prevent the shuttle 60 from being slid thereover by the user such that the sheath 60 is retained upon the shuttle 20. The flared distal tip 24a may be disposed upon the shuttle 20 with a manufacturing step performed after the sheath 60 is initially slid over the shuttle 20. The flared distal tip 24a may be formed by locally heating the material forming the shuttle 20 to allow the distal tip 24a to be flared or expanded outward. In other embodiments, the flared distal tip 24a may be monolithically formed with the shuttle 20, e.g. the shuttle 20 may be molded with the flared distal tip 24a. In those embodiments, the handle 80 may be a separate component from the shuttle 20 and fixed thereto (with adhesive, press fit, fasteners or the like) after the sheath 60 is slid over the proximal end portion 22 of the shuttle 20.

In use, a biological sample M is disposed upon the bottom surface 42 of the trough 40. Upon placement of the biological sample, the professional slides the sheath 60 coaxially over the shuttle 20 until the central portion 64 of the shuttle 60 is in registry with the trough 40. As the sheath 60 is slid over the shuttle 20, the first and second ends 65, 66 are expanded outward as they pass over the bulge portions 30 disposed upon the shuttle 20. Specifically, as can be appreciated with reference to FIG. 2, the first end 65 of the sheath 60 is slid over the distal end portion 24 of the shuttle 20 and coaxially moved over the shuttle 20 until the tip of the proximal end 65 contacts the step 82 formed by the intersection with the handle 80, or otherwise is properly positioned with respect to the shuttle 20. As the proximal end 65 is first pulled over the distal bulge 30, the proximal end 65 is expanded radially outward due to the larger outer diameter of at least a portion of the bulge 30 than the inner diameter of the proximal end 65.

As the sheath 60 is continued to be pulled over the shuttle 20, the proximal end 65 clears the distal bulge 30 such that the central member 64 is disposed over the distal bulge 30. With additional movement in the same direction, the proximal end 65 of the sheath 60 encounters the proximal bulge 30 and the distal end 66 encounters the distal bulge 30a. The respective bulges 30 urge outward expansion of the respective end portion 65, 66 to allow the sheath 60 to be pulled over the shuttle 20. The tight fit between the shuttle 20 and sheath 60 at the bulge portions 300a and the forced expansion of the first and second ends 65, 66 of the second member cause surface to surface contact between the bulges 30 and the end portions of the sheath 60 around substantially the entire outer circumference of the respective bulge 30, which substantially prevents fluid flow between the respective bulge 30 and the inner surface of the sheath 60.

Upon proper alignment of the sheath 60 with respect to the shuttle 20, the central portion 64 of the sheath 60 may be crimped inwardly with tweezers or another tool configured to crimp the surfaces of the central member toward each other. The crimping of the central member 64 establishes surface contact between the biological sample M disposed upon the bottom surface 42 of the trough 40 and the central member 64 to allow for convection heat transfer therebetween, for highly efficient and rapid heat transfer ultimately between the biological sample M and the respective cooling or warming fluid. After the central portion 64 is crimped, the combined shuttle 20 and sheath 60 are dipped or lowered into the cryogenic fluid, such as liquid nitrogen, for long term storage.

As the assembled device 10 is disposed into the cryogenic fluid, the substantially cooler cryogenic fluid rapidly receives heat from the biological sample M through the central portion 64 (and the remainder of the device 10), such that the biological sample M and the device 10 cools to substantially the same temperature as the cryogenic fluid in a very short time period, such as substantially less than one second. The method of assembling the device 10 (i.e. placing the biological sample M upon the bottom surface 42 of the trough 40 and sliding the sheath 60 over the shuttle 20 until the central member 64 is in registry with the trough 40, and then crimping the central member 64) is configured to be performed in a relatively short time period (such as less than 10 seconds) to minimize the amount of time that the biological sample is at normal room temperature in the presence of vitrification media, which may damage the biological sample M if at room temperature for extended time periods. The speed of assembling the device 10 and vitrifying the sample using the device 10 is possible because the device 10 may be assembled without the use of any external tools and without requiring that any of the ends of the device be heat fused. In addition to the time savings, the device 10 does not require heat fusing one or both ends of the device, which if done improperly could damage the device or even the sample if performed improperly, and could also cause personal injury. As discussed below, the ease of assembly of the device 10 (and specifically the ability to assemble the device 10 without requiring external tools or a heat source) allows for receipt of a biological sample M and assembly and placement into cryogenic fluid in a substantially more rapid manner than would be possible with conventional devices where the device must be assembled (and disassembled) with external tools and/or fused with a heat source.

When it is desired to warm the biological sample M for further manipulation or for implantation into a patient, the device 10 is removed from the cryogenic fluid and placed into a warming bath, which may be substantially room temperature or body temperature liquid. The placement of the device 10 within the warming fluid causes rapid heat transfer to the biological sample M through the central portion 64 of the sheath 60, which heats the biological sample M (as well as the remainder of the device 10) to about the temperature of the warming fluid in a rapid manner (such as less than one second for at least the biological sample M). As heat is transferred from the warming fluid to the central member 64 and the biological sample M, the temperature of the central member 64 increases until it reaches and exceeds the transition temperature of the material, which causes the central portion 64 to automatically rebound to its nominal, non-crimped orientation, such that the central portion 64 no longer contacts the biological sample M.

After the device 10 has been disposed within the warming fluid and the central portion 64 rebounds to its nominal position, the device 10 is removed from the warming bath. The user pulls the second cylindrical portion 60 linearly away from the shuttle 20 until the trough 40 is exposed, which allows the user to remove the biological sample M therefrom and process the biological sample M as needed to remove the vitrification media therefrom and process and/or manipulate the biological sample as desired.

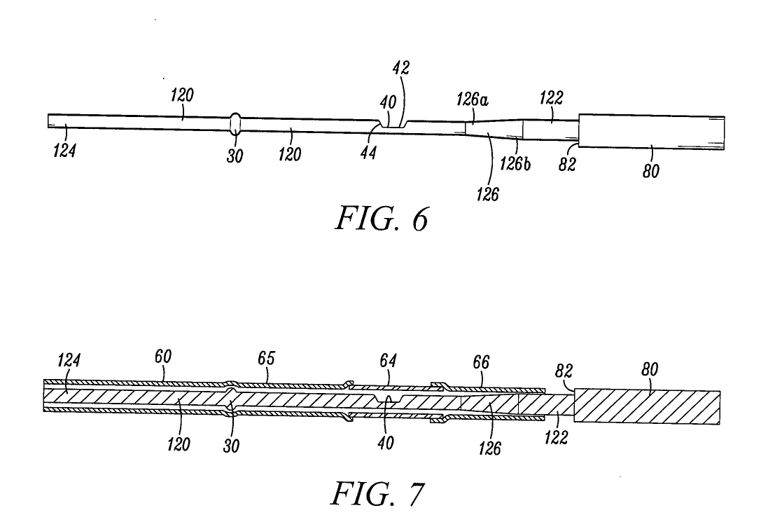

Turning now to FIG. 6, an alternate sheath 120 is provided. The shuttle 120 is constructed and operates with a sheath 60 in a generally similar manner to the device 10 discussed above. For the sake of brevity, similar components in shuttle 120 are provided with the element numbers provided in the embodiment above and reference should be made to the above discussion of those elements. The shuttle 120 operates with an elongate sheath 60 that is configured to slide about the shuttle 120 such that at least two portions of the inner diameter of the sheath 60 contact the outer surface of the shuttle 120. The shuttle 120 includes a trough 40 that is disposed within the body of the shuttle 120 and is configured to receive one or more biological samples thereupon. The trough 40 is similar to the trough 40 discussed with respect to device 10, above. The shuttle 120 additionally includes a bulge 30 disposed on a distal end 124 of the shuttle 120 proximate to the trough 40. The bulge 30 may be similar to the bulge 30 discussed with respect to the device 10, discussed above.

The proximal end 122 of the shuttle 120 additionally includes a flared portion 126 disposed on the side of the trough 40 from the bulge 30. The flared portion 126 includes an expanding outer diameter from a portion proximate the trough 40 to the proximal end 122. In some embodiments, the flared portion 126 may be linearly expanding, while in other embodiments, the flared portion 126 may expand in other geometries. The flared portion 126 includes a starting end 126a that has an outer diameter less than the inner diameter of the sheath 60, and specifically the first and second end portions 65, 66 of the sheath 60. As an exemplary embodiment, the flared portion 126 of the shuttle 120 may increase from an outer diameter of about 0.068 inches at the starting end 126a to an outer diameter of about 0.082 inches at the extended end 126b (with the inner diameter of the first end portion 65 of the sheath 60 being less than 0.082 inches). In other embodiments, the dimensions of the flared portion 126 of the shuttle 120 and the sheath 60 may be suitable for the desired size of the entire device 10 as well as the size of the biological sample M intended for use with the device 10.

The profile of the flared portion 126 expands along the length of the shuttle 120 to an outer diameter larger than the outer diameter of the first and second end portions 65, 66, such that the respective end portion 65, 66 that extends over the flared portion 126 (as the sheath 60 is slid over the shuttle 120) toward registration between the shuttle 120 and the sheath 60, such that the flared portion 126 causes radial expansion of the sheath 60 and causing surface to surface contact between the sheath 60 and the flared portion 126 of the shuttle 120, which substantially prevents fluid communication between the flared portion 126 and the sheath 60. The shuttle 120 is configured such that the opposite end portion 66 of the sheath 60 engages the bulge portion 30 upon the shuttle 120 as the first end portion 65 of the sheath 60 engages the flared portion 126 to substantially enclose the trough 40 of the device 10. The shuttle 120 may additionally include a handle 80 that is disposed upon the proximal end thereof. In embodiments where the shuttle 120 includes a handle 80, the registry between the shuttle 120 and the sheath 60 may be established when a sufficient frictional connection between the sheath 60 and the flared portion 126 of the shuttle 120 is established to prevent further linear motion of the sheath 60 with respect to the shuttle 120. In other embodiments, the sheath 60 may be in registry with the shuttle 120 when the tip of the sheath 60 contacts a step 82 between the proximal end of the shuttle 120 and the handle 80.

In use, the device using the shuttle 120 is assembled after the user places a biological specimen M upon the trough 40 and the sheath 60 is slidably disposed about the shuttle 120. Specifically, the first end 65 of the sheath 60 is placed over the distal end 124 of the shuttle 120 that includes the bulge 30 such that the first end 65 of the sheath 60 is stretched outwardly over the bulge portion 30 with continued linear movement. As the user continues sliding the sheath 60 over the shuttle 120, the first end 65 passes over the trough 40 and with continued translation slides over the proximal end portion 122 of the shuttle 120 until the first portion 65 encounters the flared portion 126. With motion of the first end 65 of the sheath 60 over the flared portion 126, the flared portion 126 urges outward radial expansion of the sheath 60, which increases the friction between the two components and the tightness of the surface to surface contact therebetween.

With sufficient motion of the sheath 60 with respect to the shuttle 120, a substantially leak tight connection is established between the sheath 60 and the flared portion 126. With the connection between the sheath 60 and the flared portion 126 established, the central portion 60 of the sheath 60 is disposed in registry with the trough 40 to allow subsequent crimping or deformation of the central portion 64 for surface contact between the central portion 64 and the biological sample M for conductive heat transfer therebetween. Similarly, as the sheath 60 is positioned with respect to the shuttle, the second end 66 of the sheath 60 contacts and is expanded by the bulge portion 30 to establish tight surface to surface contact therebetween to substantially prevent fluid communication therebetween, therefore isolating the trough 40. With the sheath 60 disposed in registry with the shuttle 120, the central portion 64 may be crimped with respect to the shuttle 120, and vitrified and cooled as discussed above with respect to the device 10.

While the preferred embodiments of the disclosure have been described, it should be understood that the disclosure is not so limited and modifications may be made without departing from the disclosure. The scope of the invention is defined by the appended claims, and all devices that come within the meaning of the claims, either literally or by equivalence, are intended to be embraced therein.

1. A cryopreservation device for storing reproductive biological material comprising:
an elongate first member extending between a distal end portion and a proximal end portion, a first bulge portion disposed around a circumference of the first member, and a trough defined within the first member, the trough being configured to receive a reproductive biological sample thereon; and
a second member with a lumen defined therethrough, the second member configured to slide over the first member, the inner diameter of the second member being similar to an outer diameter of the bulge portion to form a seal between the first and second members.

2. The cryopreservation device of claim 1, wherein the first member further comprises a second bulge portion disposed upon an opposite side of the trough from the first bulge portion.

3. The cryopreservation device of claim 1, wherein the proximal end portion of the first member is flared outward to an outer diameter larger than the inner diameter of the lumen of the second member.

4. The cryopreservation device of claim 1, wherein the second member is configured with a central portion made from a shape memory material and opposite end portions connected with the central portion are made from a material with a lower durometer than the material used for the first member.

5. The cryopreservation device of claim 4, wherein the inner diameter of each of the opposite end portions of the second member is just smaller than the outer diameter of the first bulge portion, and the opposite end portions of the second member are sufficiently flexible to deform radially outward to allow the second member to be slid over the bulge portions while establishing a relatively tight seal between the bulge portions and the second member.

6. The cryopreservation device of claim 4, wherein the central portion of the second member is configured to align in registry with the trough when the second member is slid concentrically over the first member.

7. The cryopreservation device of claim 1, wherein the first member comprises a handle disposed at the proximal end thereof, wherein the handle has an outer diameter larger than the outer diameter of the remainder of the first member that forms a step at a transition between the handle and the remainder of the first member, wherein an end of the second member contacts the step when the second member is fully slid over the first member establishing a registry between the first and second members.

8. The cryopreservation device of claim 1, wherein the trough has a bottom surface that is proximate a longitudinal central axis of the first member.

9. The cryopreservation device of claim 8, wherein the trough comprises two side surfaces on opposite ends of the bottom surface, wherein one or both of the two side surfaces are disposed at an acute angle with respect to the longitudinal axis of the first member.

10. The cryopreservation device of claim 8, wherein the bottom surface of the trough has a roughened surface.

11. The cryopreservation device of claim 1, further comprising a second bulge portion disposed upon the first member on an opposite side of the trough from the first bulge portion, wherein the second member is configured to slide over the first member wherein engagement between the first and second bulge portions and the inner surface of the second members substantially prevents fluid flow past the engagement between the respective bulge portion and the second member.

12. The cryopreservation device of claim 1, wherein each of the first and second members are substantially cylindrical.

13. The cryopreservation device of claim 1, wherein the proximal end portion is disposed between a proximal tip of the first member and the trough, and wherein the proximal end portion is longer than a length of the second member such that the second member may be disposed about the proximal end portion of the first member with the trough exposed.

14. The cryopreservation device of claim 1, wherein the trough is formed with a depth greater than a diameter of a nominal reproductive biological specimen configured to be used with the first member.

15. A method of cryogenically preserving reproductive biological material, comprising:
providing a first elongate member with a trough disposed upon an outer surface of the first elongate member, and a first bulge coaxially defined upon the outer surface with an outer diameter of the first bulge larger than an outer diameter of the first member;
depositing a reproductive biological sample upon the trough;
sliding an elongate second member over the first member to form a preservation assembly, wherein the second member has an inner diameter substantially the same as the outer diameter of the first bulge such that an inner surface of the second member makes substantially continuous contact around the circumference of the bulge portion; and
depositing the preservation assembly within a cryogenic medium.

16. The method of claim 15, further comprising removing the preservation assembly from the cryogenic medium and withdrawing the second member from the first member.

17. The method of claim 15, wherein the second member comprises a tubular shape memory portion disposed to be in registry with the trough when the second member is disposed over the first member forming the preservation assembly.

18. The method of claim 17, further comprising the step of crimping the shape memory portion when the second member is disposed over the first member, such that crimping the shape memory portion establishes contact between the shape memory portion and the reproductive biological sample for conductive heat transfer therebetween.

19. The method of claim 17, wherein the second member further comprises a flexible tubular portion connected to the shape memory portion, wherein the flexible tubular portion is disposed in registry with the first bulge portion when the second member is disposed over the first member, wherein the nominal inner diameter of the flexible tubular portion is slightly smaller than the outer diameter of the first bulge portion, and engagement between the first bulge portion and the flexible tubular portion urges outward radial expansion of the tubular portion to establish a tight seal between the first bulge portion and flexible tubular portion.

20. The method of claim 15, wherein the first elongate member further comprises a second bulge portion disposed on an opposite side of the trough from the first bulge portion.
